(19) United States
(12) Patent Application Publication
(10) Pub. No.: US 2008/0175780 A1
(43) Pub. Date: Jul. 24, 2008

(54) HYDROGEN STORAGE WITH GRAPHITE ANION INTERCALATION COMPOUNDS

(75) Inventors: Guido Peter Pez, Allentown, PA (US); Hansong Cheng, Allentown, PA (US); Alan Charles Cooper, Macungie, PA (US); Maw Lin Foo, Breinigsville, PA (US)

Correspondence Address: AIR PRODUCTS AND CHEMICALS, INC.;PATENT DEPARTMENT, 7201 HAMILTON BOULEVARD, ALLENTOWN, PA, 181951501, US

(73) Assignee: AIR PRODUCTS AND CHEMICALS, INC., Allentown, PA (US)

(21) Appl. No.: 12/015,712

(22) Filed: Jan. 17, 2008

Related U.S. Application Data
(60) Provisional application No. 60/881,212, filed on Jan. 19, 2007.

(51) Int. Cl. C01B 31/04; C01B 21/00; C07C 49/12; C07C 261/04; C01B 35/14

(52) U.S. Cl. 423/276; 423/448; 423/364; 568/412; 564/103; 423/447.1

(57) ABSTRACT

The disclosure relates to a material for reversibly storing and releasing hydrogen comprising graphite or a graphitic structure, for example, comprising an ordered graphite structure of carbon and nitrogen atoms wherein the interlayer and/or interstitial volume is occupied with at least one intercalated anionic species. While any suitable anionic species can be employed, examples of suitable species are at least one of: F⁻ (fluoride), (C≡C)²⁻ (diacetylide), and (N═C═N)²⁻. Desirable anionic species typically have a relatively high charge to volume ratio. The disclosure also relates to a material for reversibly storing and releasing hydrogen comprising ordered graphitic structures comprising at least one member selected from the group of graphite, single walled carbon nanotubes, multiwalled carbon nanotubes, graphite nanofibers, carbon nanohorns, and boron nitride. The carbon materials may incorporate substitutional nitrogen atoms in the graphite lattice at a level ranging from about 1 to about 40 atomic percent.

### CROSS REFERENCE TO RELATED PATENTS AND PATENT APPLICATIONS

The subject matter of the instant invention is related to the following U.S. patent and patent application Ser. Nos. 11/266,803; 11/437,110; 11/398,961; 11/398,965; 11/398,960. The disclosure of these patent applications is hereby incorporated by reference.

This Application claims benefit of Provisional Application No. 60/881,212, filed Jan. 19, 2007. The disclosure of this Provisional Application is hereby incorporated by reference.

### STATEMENT REGARDING FEDERALLY SPONSORED RESEARCH OR DEVELOPMENT

The Government has rights in this invention pursuant to Agreement Number DE-FC36-04GO14006 between the Department of Energy and Air Products And Chemicals, Inc.

### BACKGROUND OF THE INVENTION

The instant invention relates to an efficient hydrogen storage system for use with a hydrogen-powered device (e.g., a fuel cell, internal combustion engine, among others). Such storage systems require lightweight materials that are capable of interacting with molecular hydrogen via either physisorption or chemisorption. This interaction typically occurs with an appropriate strength to enable the “capture” of hydrogen to be reversible under practical operating conditions of temperature and/or pressure. The inventive materials possess a desirable combination of hydrogen adsorption enthalpy and hydrogen storage density.

Hydrogen is a widely used commodity in the chemical and petroleum processing industries. Typically it is manufactured by a reforming of natural gas and is delivered to the users' sites by pipeline, as liquid H₂ or as a highly compressed gas in cylinders. The transport of hydrogen as a cryogenic liquid or as compressed gas are capital and energy-intensive processes that result in a significantly higher cost for the delivered gas. Therefore, there has been a large research effort directed at finding lower cost alternatives, principally on developing materials that could effectively “capture” hydrogen at or near ambient conditions and release the gas as desired, at the point of use. It is widely recognized that a prototypical hydrogen storage device requires lightweight materials that are capable of interacting with molecular hydrogen via either physisorption or chemisorption. This interaction must occur with an appropriate strength (H₂ sorption heat or enthalpy) to enable the “capture” of hydrogen to be reversible under practical operating conditions of temperature and/or pressure.

Considerable research has been devoted in recent years to advanced metal hydrides (e.g. NaAlH₄ and MgH₂) and chemical hydrides (e.g. NH₃BH₃ and NaBH₄) to increase the hydrogen capacity and reversibility of these materials. Despite the promising performance of recently discovered metal hydride and chemical hydride materials, there are anticipated problems with the use of these materials in hydrogen storage systems, such as slow refilling times (e.g., a refueling station for a passenger vehicle), due to heat transfer issues. Porous hydrogen storage materials that operate by the physical adsorption of hydrogen are expected to reduce some of the hydrogen storage system engineering issues due to their lower heat of adsorption relative to most metal hydrides. Examples of hydrogen storage materials with inherent porosity that operate by the physical adsorption of hydrogen include activated carbons (U.S. Pat. No. 4,580,404), single walled carbon nanotubes (U.S. Patent Publication No. 2005/0118091 A1), and metal-organic framework materials (Collins, D. J.; Zhou, H.-C. J. Mater Chem. 2007, 17 3154). However, the known porous materials of these classes demonstrate a heat of adsorption for hydrogen that is too low for a practical storage of hydrogen at near-ambient temperatures. Other carbon materials, such as crystalline graphite, do not have large hydrogen adsorption due in part to a lack of porosity.

Chemical intercalation of graphite with alkali metals has been demonstrated to vastly increase the hydrogen adsorption capacity by rendering the crystalline graphite porous through intercalation and separation of the graphene layers as well as increasing the hydrogen affinity of the graphite through an increased electron density from the alkali metals. The resulting materials are known as a graphite intercalation complexes or Graphite Intercalation Compounds (GIC). For example, the second-stage graphite intercalation complex KC₂₄ has been shown to adsorb two hydrogen molecules/potassium at 77 K corresponding to 1.2 wt. % hydrogen (Watanabe, K.; Soma, M.; Onishi, T.; Tamaru, K. Nature 1971, 233, 160). Electron transfer from potassium to the graphene layers in this “donor type” graphite intercalation complex increases the heat of adsorption from 1 kcal/mol in native graphite to ca. 2.3 kcal/mol in KC₂₄ (Terai, T.; Takahashi, Y. Synth. Met. 1989, 34, 329; Yea, N.; Enoki, T.; Salamanca-Riba, L.; Dresselhaus, G. Mater. Res. Soc. 1986, 56, 467). However, the heat of adsorption in the known donor type GIC is not sufficient for the effective hydrogen storage at near ambient temperatures. A higher heat of adsorption is desired for hydrogen storage at near ambient temperatures. The examples of KC₂₄ and CsC₂₄ are donor type GIC's where the graphite accepts a certain amount of negative charge from the intercalating species. In contrast, acceptor GIC's are prepared under conditions where the graphite is at least partially oxidized so that it retains a certain amount of positive charge, which in the resulting intercalation compound is balanced by the negative charge on the anion. Commonly known acceptor type GIC's are those intercalated with anions such as halides, nitrate, and sulfate. The graphite in these acceptor GIC's may be viewed as being an extended (non-localized) cation. There are no known examples in the literature of acceptor type GIC's with demonstrated hydrogen adsorption properties.

Graphite fluoride represents a widely studied subset of acceptor GIC's (Watanabe, N.; Nakajima, T.; Touhara, H. Graphite Fluorides; Elsevier: Amsterdam, 1988; Fluorine-Carbon and Fluoride-Carbon Materials; Nakajima, T., Ed.; Marcel Dekker: New York, 1995). While most acceptor GIC's are ionic compounds with a high level of change transfer from graphene to the intercalate anions to generate (graphene)⁺ X⁻, intercalated fluorine can be in the form of ionic (graphene)⁺ F⁻, covalent (graphene)-F, and intermediate mixed ionic-covalent or “semi-ionic” structures depending on the fluorine concentration in the materials. The more ionic fluorine intercalates predominate at low levels of fluorine where the graphite retains its planar structure. At high fluorination levels non-planar, cyclohexane-type structures are formed. (Dresselhaus, M. S.; Endo, M; Issi, J.-P. Physical Properties of Fluorine- and Fluoride-Graphite Intercalation Compounds; and Nakajima, T. Synthesis, Structure and Physicochemical Properties of Fluorine-Graphite Intercalation Compounds in Graphite Fluorides; Elsevier: Amsterdam, 1988; Fluorine-Carbon and Fluoride-Carbon Materials; Nakajima, T., Ed.; Marcel Dekker: New York, 1995, chapter 1 (pgs. 1-31) and chapter 4 (pgs. 95-112)). The presence of an ionic fluoride, F⁻, component in graphite fluoride materials can be determined by solid state NMR (Giraudet, J.; Dubois, M.; Guerin, K.; Delabarre, C.; Hamwi, A.; Masin, F. Solid-State NMR Study of the Post-Fluorination of (C_{2.5}F)ₙ Fluorine-GIC. J. Phys. Chem. B. 2007, 111(51),14143).

The synthesis of BF₄ intercalated graphite, CₓBF₄, is achieved by exposing a synthetic graphite powder to a mixture of fluorine (F₂) and boron trifluoride (BF₃) gas at room temperature and the decomposition of intercalated BF₄ to fluoride can be accomplished by mild heating under vacuum (Nikonorov, Yu. I. Catalytic effect of boron trifluoride on graphite oxidation by fluorine. Kinetika i Kataliz 1979, 20(6), 1598).

Sweany et al. reported an infrared characterization of simple adducts of H₂ with cesium fluoride ion pairs in a solid argon matrix at 10 K (Sweany, R. L. et al. Inorg. Chem. 1997, 36, 2523). The normally IR silent H₂ molecule is sufficiently perturbed by the salt, giving rise to spectra which are interpreted as arising from H₂ and CsF adducts, with as many as three hydrogen molecules per cesium fluoride. The data suggested that the H₂ interacts primarily with the fluoride anion and based on shifts in the H—H stretching frequency showing a lengthening of the H—H bond, this interaction is proposed to be donation of electron density from the fluoride anion to the H₂ antibonding orbitals.

The disclosure of the previously identified patents and patent applications is hereby incorporated by reference.

### BRIEF SUMMARY OF THE INVENTION

This invention provides a solution to the ineffectiveness of conventional solid hydrogen storage materials by providing a material for reversibly storing and releasing hydrogen comprising a graphitic structure, for example, comprising carbon, an ordered (or partially ordered) graphite structure of carbon or carbon and nitrogen atoms wherein the interlayer and/or interstitial volume is occupied with at least one of intercalated anionic species. While any suitable anionic species can be employed, examples of suitable species comprise at least one of: F⁻ (fluoride), (C═C)²⁻ (diacetylide), and (N═C═N)²⁻. For effective hydrogen storage, the material provides both the porosity and an affinity for hydrogen as measured by the heat of adsorption of hydrogen. Without being bound by theory or explanation, it is believed that the affinity of the material for hydrogen arises from an interaction of hydrogen with the intercalated anions. It is also believed that the graphitic host material provides the positive charge that is necessary for an overall charge neutral structure.

In one aspect of the invention, the anionic species are characterized by a relatively high charge to volume ratio (e.g., having charge density of about 0.14 to about 0.02 electron/Å³, thus favoring an interaction with hydrogen. Larger anions (e.g., those characterized by a relatively low charge to volume ratio) such as SO₃F⁻ may additionally be employed to render more facile an access of H₂ to the active sites of the structure. Without wishing to be bound by any theory or explanation, the anionic species can also function as pillars or spacers that can increase the distance between lattices of the graphitic structure relative to the distance without the anionic species. It is also believed that the increased distance causes the structure to become more porous to molecular hydrogen thereby permitting enhanced interaction between the anionic species and hydrogen.

This invention additionally provides a material for reversibly storing and releasing hydrogen comprising ordered graphitic structures comprising at least one member selected from the group of graphite, single walled carbon nanotubes, multiwalled carbon nanotubes, graphite nanofibers, carbon nanohorns, and boron nitride. Typically, the carbon materials incorporate substitutional nitrogen atoms in the graphite lattice at a level ranging from about 1 to about 40 atomic percent.

In another aspect of the invention, the graphitic structure comprises nitrogen-containing carbon materials. Since a cationic nitrogen atom, N⁺, is isoelectronic with a carbon atom, it is believed that a nitrogen-substituted graphite will more easily stabilize a positive charge. This is advantageous for forming the acceptor GIC's, (graphite)+X⁻ (where X⁻ is an anion), of the present invention. Nitrogen-containing carbon materials may be prepared by any suitable method such as a high temperature pyrolysis of organonitrogen compounds in an inert atmosphere, chemical vapor deposition (CVD) synthesis methods using organonitrogen precursors and metal catalysts, and by N-atom incorporation from plasma sources (Nakajima T.; Koh M. Synthesis of highly crystalline carbon-nitrogen layered compounds by CVD using nickel and cobalt catalysts. Carbon 1997, 35, 203; Ewels, C. P.; Glerup, M. Nitrogen Doping in Carbon Nanotubes. Journal of Nanoscience and Nanotechnology 2005, 5, 1345).

This invention additionally provides a process for reversibly storing and releasing hydrogen comprising the steps of: i) providing a vessel comprising a graphitic structure comprising carbon and nitrogen atoms wherein at least one of the interlayer and interstitial volume of the structure is partially occupied with at least one of intercalated anionic species; ii) introducing hydrogen into the vessel while increasing the pressure to a sorption pressure and/or decreasing the temperature to a sorption temperature whereby the hydrogen is absorbed by the hydrogen storage material; and iii) discharging the hydrogen from the vessel by decreasing the pressure from the sorption pressure to a desorption pressure and/or increasing the temperature to a desorption temperature whereby the hydrogen is desorbed by the hydrogen storage material. This process can be repeated in order to reversibly store and release hydrogen.

### BRIEF DESCRIPTION OF SEVERAL VIEWS OF THE DRAWINGS

FIG. 1 illustrates the geometry, bond angles, and interatomic distances (Angstroms): (a) F⁻ with 3 H₂ molecules, (b) (CN)⁻ with 3 H₂ molecules, (c) (C≡C)²⁻ with 4 H₂ molecules, (d) (C═N═C)²⁻ with 4 H₂ molecules.

FIG. 2 illustrates an equilibrated unit cell for H₂ adsorption in the nitrogen containing graphite fluoride intercalation compound with 24 H₂/unit cell (C₂₄N₈F₈H₄₈).

FIG. 3 illustrates the calculated radial distribution functions of C—F, N—F, F—H and C—H distances for H₂ adsorption in the nitrogen containing graphite fluoride intercalation compound with 24 H₂/unit cell (C₂₄N₈F₈H₄₈). The x-axis units are Å (angstroms) and the y-axis units are arbitrary.

FIG. 4 illustrates an ab initio molecular dynamics simulation for H₂ adsorption in nitrogen containing graphite fluoride intercalation compound with 24 H₂/unit cell (C₂₄N₈F₈H₄₈) at times of 1.06 picoseconds (ps), 1.50 ps, 2.03 ps and 2.54 ps.

FIG. 5 is a plot of hydrogen adsorption isotherms on graphite fluoride at selected hydrogen pressures ranging between 0-1500 psia and temperatures of 273, 298, and 313 K.

FIG. 6 is a plot of the calculated isosteric heat of hydrogen adsorption on graphite fluoride using isotherm data collected at temperatures of 273, 298, and 313 K.

FIG. 7 is a plot of hydrogen adsorption isotherms on activated carbon at selected hydrogen pressures ranging between 0-1600 psia and temperatures of 273, 298, and 323 K.

FIG. 8 is a plot of the calculated isosteric heat of hydrogen adsorption on activated carbon using isotherm data collected at temperatures of 273, 298, and 323 K.

### DETAILED DESCRIPTION OF THE INVENTION

Despite conceptual calculations of the interaction of hydrogen with the fluoride ion and other anions in the gas phase (as illustrated in Example 1), anions cannot exist alone in the gas phase. It is also unrealistic to expect that a nonporous crystalline fluoride salt compound such as cesium fluoride will have its F⁻ ions readily accessible to hydrogen. The practical problem then lies in devising solid state highly ionic compositions where the fluoride (and other anions) are both accessible to H₂ and retains most of its charge. Anhydrous tetraalkylammonium fluoride salts such as tetramethylammonium fluoride, (CH₃)₄N⁺F⁻, are highly reactive sources of nucleophilic (naked) fluoride, as for instance demonstrated by its facile reactivity with CO₂, even as a solid, to form the fluorocarbonate (FCO₂⁻) salt (Seppelt, K. et al. Angew. Chem. Int. Ed. Engl. 1995, 34, 1858). This adduct has a significant dissociation pressure at room temperature and upon loss of CO₂ a highly dispersed form of the fluoride salt is retained.

Graphite fluoride intercalation compounds are known. Because of the unique nature of fluorine, as an intercalate in graphite it can exist in an ionic (as F⁻) covalent (as C—F) or in a mixed ionic, covalent or “semi-ionic” structure, depending on the concentration of fluorine (Sato, Y et al. On the so-called “semi-ionic” C—F bond character in fluorine—GIC. Carbon 2004, 42, 3242; Amine, K.; Nakajima, T. A new stage-2 graphite intercalation compound: CF₂ with nestled fluorine atoms. Carbon 1993, 31, 553).

An ideal “non-interacting” cation would be in the form of a porous solid-state material that is associated with charge balancing, free or “naked” anions such as fluoride. We consider here also graphite intercalation complexes in which some of the carbon atoms in graphite are replaced by an isoelectronic N⁺ atom, and the overall resulting positive charge of the graphitic structure is balanced by the anions intercalated in the lattice. For example, a specific composition comprises (C₆N₂)ₙ²ⁿ⁺ 2nF⁻, where two of the eight carbon atoms in the unit cell of graphite are replaced by nitrogen. A nitrogenous graphite composition (composition C₅N) can be obtained by reacting pyridine with Cl₂ at 700° C. (J. Kouvetakis, R. Kaner, M. Sattler & N. Bartlett J. Chem. Soc. Chem. Comm. 1986, 1758). It reportedly has a bright metallic luster and it is implied that an intercalation compound might be accessible by reaction with the oxidant S₂O₆F₂ (J. Kouvetakis, T. Saski, C. Shen, R. Hagiwara, M. Lerner, K. M. Krishnan & N. Bartlett Synthetic Metals 1989, 34, 1). A similar process of reaction of a graphitic material with a suitable oxidant or an oxidant and an anionic species might also be applicable to the syntheses of other anion-intercalated graphitic materials.

The synthesis of Cₙ⁺SO₃F⁻ from the reaction of graphite with S₂O₆F₂ (S. Karunamithy, et al. Structure and bonding in graphite fluorosulfate derivatives. Journal de Chimie Physique de Physico-Chemie Biologique 1986, 81, 897), and SO₃F⁻ containing B/C/N materials (Novel aspects of graphite intercalation by fluorine and fluorides and new B/C, C/N and B/C/N materials based on the graphite network. Synthetic Metals 1989, 34, 1) have been described.

For nitrogen-modified graphitic structures containing fluorine there is evidence for a “semi-ionic” C—F bond (Groult, H. et al. Structural analysis and electrochemical characteristics of new CₓNF_{z} compounds in aprotic media. Electrochemica Acta 1999, 44, 3521). In the graphite and nitrogen-substituted graphitic materials of this invention, F⁻, C≡C²⁻, N═C═N²⁻, and other anions can be intercalated in the lattice as the free ions, bound to the cationic graphitic lattice mainly by electrostatic forces. As exemplified by Examples 1-5, these relatively free anions are capable of a reversible interaction with hydrogen. Thus, for H₂ storage predominantly “largely ionic” GIC of the acceptor type are useful.

Graphite and nitrogen modified graphitic structures intercalated with predominantly ionic anions of general formula: Cₙ⁺A⁻ and (CNₓ)ₙ⁺A⁻ where A⁻ comprises at least one intercalated anion of high volumetric change density may be prepared starting from predominantly ionic intercalation compounds such as Cₙ⁺AsF₆⁻ and Cₙ⁺SbF₆⁻ and exchanging the anions with fluoride, F⁻, using “naked fluoride” sources such as (CH₃)₄N⁺F⁻ in a solvent medium: Cₙ⁺SO₃F⁻+(CH₃)₄N⁺F⁻→Cₙ⁺F⁻+(CH₃)₄N⁺SO₃F⁻. This ion exchange process may be conducted to about 80 to about 90% of completion. It is believed that such a process can enable some of the larger anions to be retained in the structure and act as pillars that separate the layers of the graphite lattice thereby providing increased volume for a more facile access of hydrogen to the smaller fluoride active sites.

Intercalation compounds of (C═C)²⁻ and (N═C═N)²⁻ may be prepared by an exchange of salts of these species in a solvent medium with acceptor type graphite intercalation compounds and nitrogen-containing acceptor type graphite intercalation compounds.

In order to demonstrate the effectiveness of the instant invention, a series of calculations based on ab initio density functional theory of hydrogen adsorption in several graphitic compounds intercalated with anionic species were conducted (Examples 1-5). Without wishing to be bound by any theory or explanation, it is believed that donation of electron density from an anion to hydrogen can increase the attractive interaction. These calculations were based upon three representative anionic species with highly localized negative charge (i,e., strong Lewis bases), F⁻, (C≡C)²⁻ and (N═C═N)²⁻ intercalated in graphitic compounds as the adsorbents for H₂ storage. The graphitic compounds are graphite and graphitic materials in which some of the carbon atoms in graphite are replaced by an isoelectronic N⁺ atom, and the overall resulting positive charge is balanced by the anions intercalated in the lattice. To ascertain the strength of the interaction between H₂ and an anion, we first performed calculations on their interaction energies in the gas-phase at various levels of theory. Subsequently, ab initio molecular dynamics (MD) simulations were performed to understand the dynamic behavior of the intercalated species, including H₂ molecules, in the GIC lattice and to estimate the adsorption energies at ambient temperature.

The electronic energy was calculated with the periodic DFT under the local density approximation with the Perdew-Zunger exchange-correlation functional using a plane-wave basis set, while the MD simulation was performed in the constant NVE canonic ensemble using the Nosé thermostat for temperature control. Ultrasoft pseudopotentials were used to describe the core electrons with cutoff energies of 425 eV for F containing compounds and 348 eV for other anion-intercalated materials. The Brillouin zone integration was performed using the Monkhorst and Pack points of 2×2×4 for the F⁻-intercalated graphitic material and 1×1×3 for the other materials. Structural equilibration was performed by optimizing all the atoms in the unit cells and the cell parameters using the conjugated gradient and quasi-Newton's algorithms. The forces acting on the atoms, derived from the ground state electronic energies, were calculated using the Hellmann-Feynman theorem at each time step and are subsequently utilized in the integration of Newton's equation of motion. The mass of tritium was used for H, which allows us to use a time step of 1 femtoseconds to simulate the dynamic systems for up to 5 picoseconds at room temperature. While the time dependent properties are subject to change due to the isotopic effect, the time-independent behavior will remain the same. All MD simulations were performed at simulation temperature of 300 K. The software described above and elsewhere in the instant description is commercially available and was employed in accordance with conventional methods.

To ascertain the interactions between H₂ and F⁻, (C≡C)²⁻ and (N═C═N)²⁻, we first calculated the H₂ interaction energies with these species in the gas phase with commercially available Jaguar 4.1 and Gaussian 03 program suites. The calculations were performed using density functional theory (DFT) under the generalized gradient approximation (GGA) using the B3LYP exchange-correlation functional. To adequately describe the anionic species, a variety of large basis sets augmented with polarization functions and diffuse functions were used in the calculations. Full geometry optimization was performed without symmetry constraints. Basis set superposition error was estimated for H₂ on F⁻ and found to be negligible. In addition, the calculated DFT results were compared with CCSD and CCSD(T) calculations.

Cheng et al. have shown that both the alkali metal cations and H₂ in certain donor type graphite intercalation compounds (e.g. KC₂₄) are highly mobile with fluid-like dynamics (Cheng, H.; Pez, G.; Kern, G.; Kresse, G.; Hafner, J., J. Phys. Chem. B. 2001, 105, 736). In order to describe the structure and energetics of the adsorbed molecules in the GIC, we performed ab initio molecular dynamics simulations at room temperature using a commercially available VASP (Vienna Ab initio Simulation Program) package. The unit cells used in these simulations were chosen based on the following considerations: i) the intercalated graphitic compounds were designed to be first stage GIC in order to gain the maximum possible H₂ storage capacity; ii) to maintain charge neutrality, the number of nitrogen atoms doped in the graphitic lattice is the same as the number of anionic species intercalated between the graphitic sheets multiplied by the absolute charge; iii) the N atoms are assumed to be distributed evenly in the graphite sheets and the intercalated anions were placed near the N atoms prior to geometry optimization; iv) H₂ molecules were initially placed at least 2.5 Å away from anions as well as from the graphitic sheets; and v) to computationally account for the high mobility of the anions and H₂ molecules in the lattice, a relatively large unit cell is required.

The unit cell of the fluoride GIC contains 24 carbon (C) atoms and 8 nitrogen (N) atoms. Based upon our gas-phase calculations, we concluded that each F⁻ will interact strongly with three H₂ molecules. Thus, eight F⁻ ions together with 24 H₂ molecules were introduced in the interlayer space, yielding a hydrogen storage capacity of about 8.0 wt. %. For the larger (C≡C)² and (N═C═N)²⁻ anions, we expanded the unit cell by 2×2 from the cell used for the F⁻-intercalated graphitic compound along the graphite plane to accommodate free motion of the anionic species. In the calculation of the (C≡C)²⁻ and (N═C═N)²⁻ complexes, there were 112 carbon atoms and 16 nitrogen atoms forming the graphene sheet of the unit cell. Based on the gas-phase computational results, each (N═C═N)²⁻ or (C≡C)²⁻ is capable of interacting substantially simultaneously with 4 H₂ molecules. Thus, there were 8 (N═C═N)²⁻ and (C≡C)²⁻ anions placed together with 32 H₂ molecules in the intercalation layers of the two simulations. These unit cells lead to calculated hydrogen storage capacities for the (N═C═N)²⁻ and (C≡C)²⁻ GIC of about 3.28 wt. % and about 3.50 wt. %, respectively.

Anion Compositions. One aspect of the invention relates to I compositions of GIC's and nitrogen-comprising graphite intercalation compounds that comprise anions having a high volumetric charge density (electron/Å³) and typically also a high charge to mass ratio (electron/amu). The volumetric charge density relates to the affinity of the anion for molecular hydrogen. As shown by the calculations in the instant invention, a high volumetric charge density favors a strong interaction with H₂. With respect to the charge to mass ratio, a relatively “light” anion leads to a high gravimetric storage capacity. Data for selected gas phase anions as calculated using the quantum mechanics methodologies B3LYP/6-311+G* using the cited Jaguar 4.1 and Gaussian 03 program suites, is found in Table 1.

TABLE 1
Table 1. Calculated charge volumetric density and weight
density of anions (molecules with the specified charge).

| | Charge | Volume | Weight | \|Q\|/V | \|Q\|/W |
|---|---|---|---|---|---|
| Molecule | (Q) | (Å³) | (amu) | (electron/Å³) | (electron/amu) |
| O | −2 | 14.71 | 16 | 0.135962 | 0.125 |
| NH | −2 | 18.12 | 15 | 0.110375 | 0.133 |
| S | −2 | 25.06 | 32 | 0.079808 | 0.0625 |
| F | −1 | 14.53 | 19 | 0.068823 | 0.052632 |
| OH | −1 | 17.01 | 17 | 0.058789 | 0.058824 |
| CN₂ | −2 | 40.4 | 40 | 0.049505 | 0.05 |
| NHLi | −1 | 21.10 | 21.94 | 0.047393 | 0.045579 |
| C₂ | −2 | 43.53 | 24 | 0.045945 | 0.083333 |
| HF₂ | −1 | 21.88 | 21.01 | 0.045704 | 0.047587 |
| CO₃ | −2 | 45.62 | 60 | 0.04384 | 0.033333 |
| O₂ | −1 | 31.998 | 32 | 0.031252 | 0.03125 |
| CN | −1 | 34.57 | 26 | 0.028927 | 0.038462 |
| BF₄ | −1 | 53.29 | 86.81 | 0.018765 | 0.011519 |
| SO₃F | −1 | 53.9 | 99.06 | 0.018553 | 0.010095 |
| AsF₆ | −1 | 78.23 | 188.91 | 0.012783 | 0.005294 |
| SbF₆ | −1 | 93 | 235.74 | 0.010753 | 0.004242 |

The volumetric charge density diminishes in the order of: O²⁻>NH²⁻>S²⁻>F⁻>OH⁻>CN₂²⁻>NHLi⁻>C₂²⁻>HF₂⁻>CO₃²⁻>O₂⁻>CN⁻>>BF₄⁻>>SO₃F⁻>AsF₆⁻>SbF₆⁻. Anions suitable for the instant invention can comprise those having a charge density that is greater than about 0.02 electron/Å³, or in some cases having a charge density that is greater than about 0.045 electron/Å³, and a molecular weight of less than about 50 amu. It will be noted that AsF₆⁻, SbF₆⁻ and SO₃F⁻ which are anions of relatively large volume and very low charge volumetric density may be employed as relatively non-H₂ interactive pillars for separating the graphite layers thereby allowing a more facile access of H₂ to the active sites of the intercalated structures.

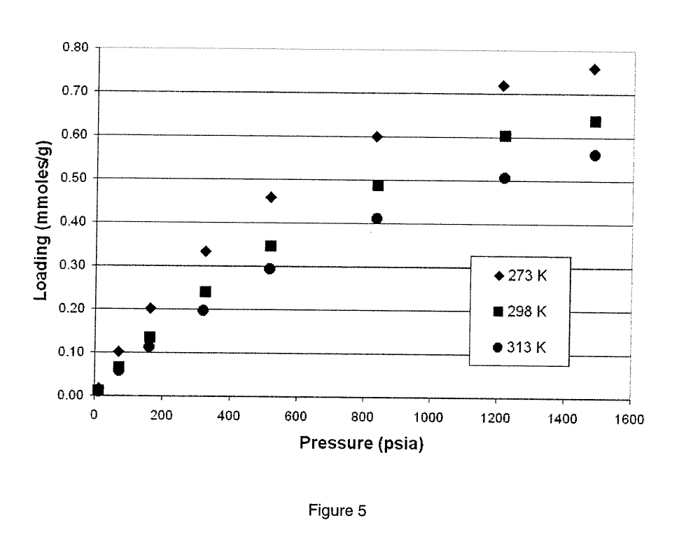

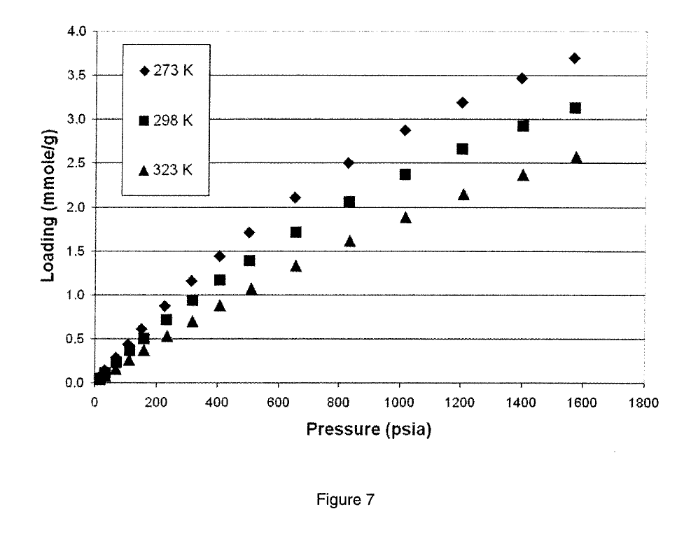

One aspect of the invention relates to methods for reversibly storing and releasing hydrogen from the inventive graphitic structure. The material is typically contained in a vessel that is equipped with the means for an entry and discharge of hydrogen at variable pressure and flow conditions. These methods provide processes for storing hydrogen using a material comprising the graphitic structure as a reversible storage media for hydrogen in: (a) a H₂-pressure-swing, (b) temperature-swing and (c) combined temperature and H₂-pressure swing processes. These processes are all reversible, and are repeated by charging at the adsorption temperature, or adsorption pressure, or adsorption temperature and adsorption pressure, and discharging at the desorption temperature, or desorption pressure, or desorption temperature and desorption pressure, for temperature swing, pressure swing, and temperature and pressure swing processes, respectively. It is not required that the hydrogen stored in the vessel comprising the graphitic material be fully charged with the maximum amount of hydrogen possible or fully discharged of all of the stored hydrogen during every cycle of the process. Desorption and adsorption temperatures and/or desorption and adsorption pressures between predetermined ranges may be useful for partial charging and partial discharging of the vessel. When the vessel containing the storage material is charged with hydrogen, the gas is at least partially adsorbed or taken up by the material to an extent that can be determined from its H₂ adsorption isotherm (for example as shown in FIGS. 5 and 7), the H₂ gravimetric loading as a function of pressure. The contained hydrogen exists as both adsorbed and free H₂ in the gas phase. The capacity of the adsorbent for hydrogen maybe sufficiently large to accommodate more hydrogen in a vessel comprising the hydrogen storage material than an empty vessel of the same volume. As a result, a desirable amount of hydrogen may be stored in the vessel (i.e., a vessel containing the storage material), at a lower pressure than was possible with an empty vessel. Additional details relating to methods for reversibly storing and releasing hydrogen can be found in U.S. Patent Publication No. 2005/0118091 A1; the disclosure of which is hereby incorporated by reference.

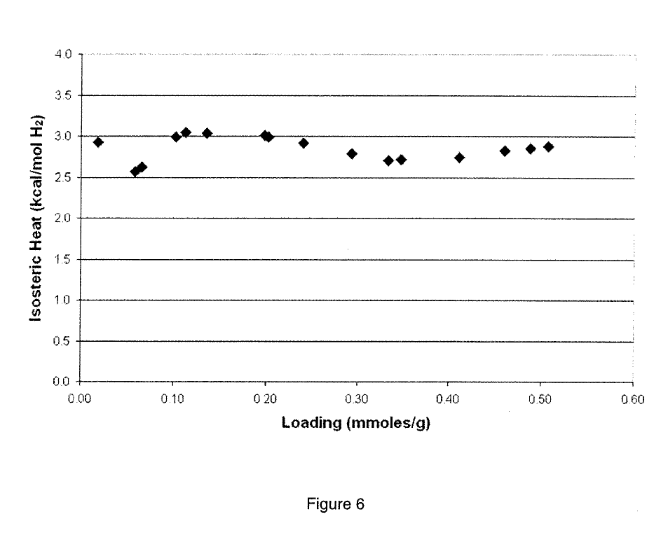

In order to determine the utility of anion graphite intercalation compounds for hydrogen storage, hydrogen adsorption isotherms were measured at different temperatures for a graphite fluoride intercalation compound as described in Example 6. The H₂ adsorption isotherms are indicative of a physical adsorption of hydrogen, defined here as a process where the hydrogen is adsorbed non-dissociatively, with retention of the H—H bond. From the isotherm data, hydrogen storage capacities and H₂ adsorption heats as function of hydrogen adsorption capacity were calculated; results for the latter are shown in FIG. 6. The isosteric heat of adsorption can be derived using experimental adsorption isotherms determined at a minimum of two temperatures (Gregg, S. J.; Sing, K. S. W. In “Adsorption, Surface Area and Porosity” second edition, Academic Press, 1982, p. 13-18). Using isotherms at several temperatures, the relationship of equilibrium pressure and temperature at identical coverages can be plotted from the isotherms for a series of temperatures. For two temperatures, T₁ and T₂, and the corresponding equilibrium pressures p₁ and p₂, an amount of gas adsorbed nₐ (i.e. the gas “coverage”), the isosteric heat (q^{st}) can be calculated from the following equation:

q^{st}=[RT₁T₂/(T₂−T₁)](ln p₂−ln p₁)nₐ
where R=the gas constant.

The following examples are presented to better illustrate certain aspects of the present invention and do not limit the scope the claims appended hereto.

### EXAMPLE 1

### Calculation of Hydrogen Interactions with Anions in the Gas Phase

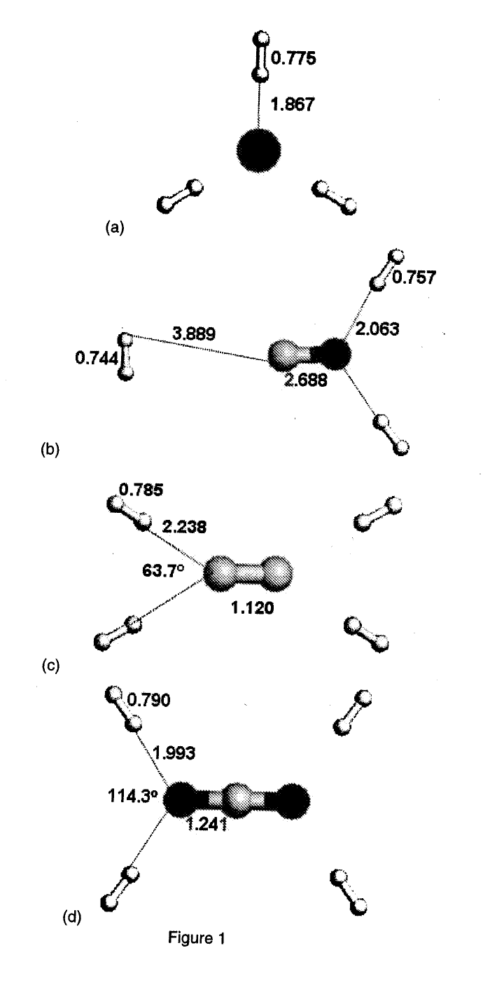

To ascertain the interactions between H₂ and the anion intercalated graphitic compounds, we calculated the interaction energies between H₂ and gas phase anions. FIG. 1 illustrates the fully optimized structures of H₂ adsorption on the anionic species calculated at the B3LYP/6-311++G** level. Table 2 lists the calculated H₂ adsorption energies obtained at various levels of theoretical methods. The basis set superposition error for H₂ interaction with F⁻ calculated with the counter poise method accounts for less than about 0.1% of the interaction energy and is thus negligible in this case. In the optimized structure for hydrogen interacting with cesium fluoride, two H₂ molecules are associated with the F ion and one H₂ is associated with the Cs⁺ ion.

TABLE 2
Table 2. The calculated average H₂ adsorption energy on fully optimized
anionic species
(unit: kcal/mol H₂) for a variety of computational methods.

| | | B3LYP/ | CCSD/ | |
|---|---|---|---|---|
| | B3LYP/6- | cc- | 6- | |
| | 311++** | pvtz(-f)++ | 311++** | LACVP++ |
| F⁻ . . . H₂ | −7.95 | −7.32 | | — |
| F⁻ . . . 2H₂ | −7.15 | −6.47 | | — |
| F⁻ . . . 3H₂ | −6.45 | −5.81 | −5.03 | — |
| H₂ . . . ⁻C≡C⁻ . . . H₂ | −7.15 | −7.53 | | — |
| 2H₂ . . . ⁻C≡C⁻ . . . 2H₂ | −6.02 | −6.00 | −3.09 | — |
| 2H₂ . . . ⁻N═C═N⁻ . . . | −9.10 | −8.42 | — | — |
| 2H₂ | | | | |
| CsF . . . H₂ | — | — | — | −5.22 |
| CsF . . . 2H₂ | — | — | — | −4.82 |
| CsF . . . 3H₂ | — | — | — | −3.90 |

### EXAMPLE 2

### Calculation of Hydrogen Interactions with a Nitrogen Containing Graphite Fluoride Intercalation Compound

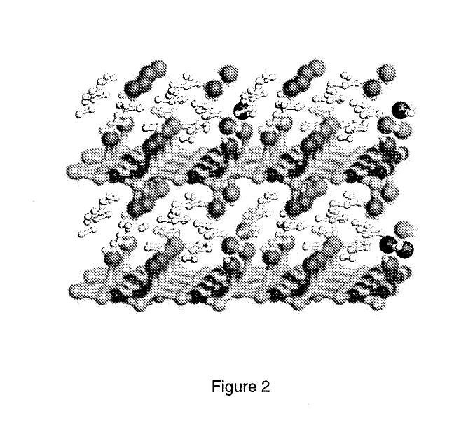

The unit cells selected to simulate H₂ adsorption in the nitrogen containing graphite fluoride intercalation compound were first fully equilibrated with and without adsorbed H₂ molecules. FIG. 2 illustrates the optimized unit cell of C₂₄N₈F₈H₄₈, containing 24 molecules of adsorbed H₂. The fully optimized unit cell parameters obtained from the calculations of hydrogen interactions with the nitrogen containing graphite fluoride intercalation compound C₂₄N₈F₈ are shown in Table 3.

TABLE 3
Table 3. The calculated unit cell parameters for the nitrogen containing
graphite fluoride intercalation compound (C₂₄N₈F₈),
the nitrogen containing graphite fluoride intercalation compound
with 12 H₂/unit cell (C₂₄N₈F₈H₂₄),
and the nitrogen containing graphite fluoride intercalation
compound with 24 H₂/unit cell (C₂₄N₈F₈H₄₈).

| | a | b | c | α | β | γ |
|---|---|---|---|---|---|---|
| C₂₄N₈F₈ | 9.530 | 9.518 | 4.410 | 89.8 | 90.3 | 123.2 |
| C₂₄N₈F₈H₂₄ | 9.586 | 9.593 | 5.901 | 90.1 | 90.2 | 119.7 |
| C₂₄N₈F₈H₄₈ | 9.597 | 9.613 | 7.189 | 90.2 | 90.4 | 120.0 |

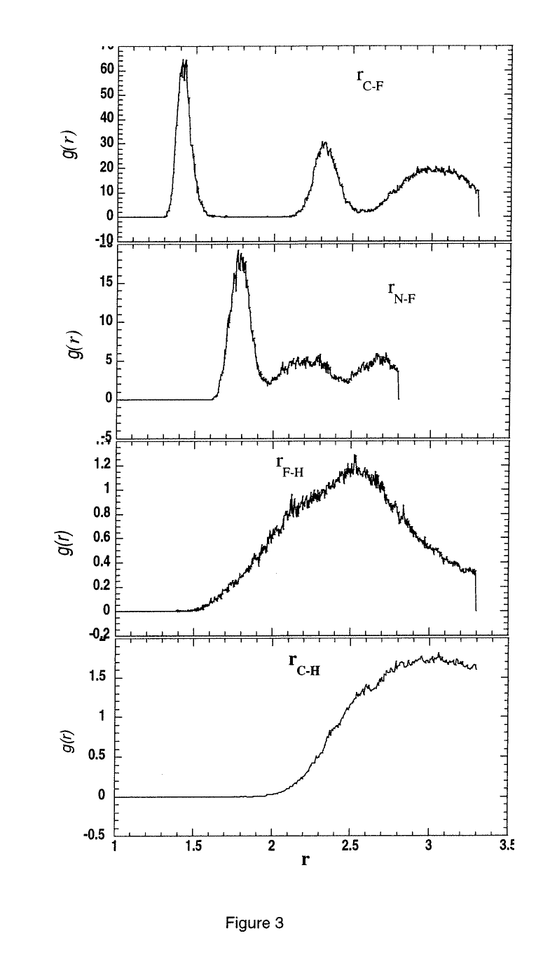

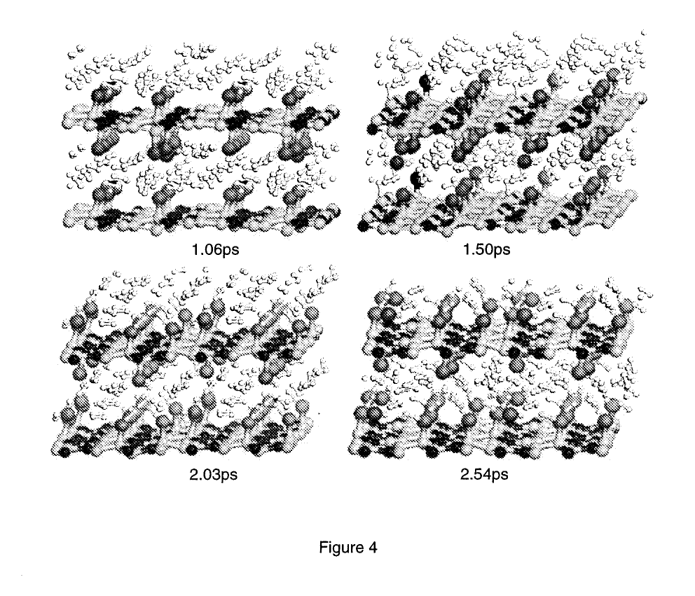

The initial molecular dynamics (MD) simulations of the nitrogen containing graphite fluoride intercalation compound showed that the optimized lattice was generally rigid at room temperature and contained distinct C—F bond linkages as observed in FIG. 2. Upon H₂ adsorption, the lattice was found to undergo expansion to accommodate the high density of H₂ molecules. The calculated radial distribution functions (RDFs) of C—F, N—F, F—H and H—C distances for the simulation of nitrogen containing graphite fluoride intercalation compound with 24 H₂/unit cell (C₂₄N₈F₈H₄₈) are illustrated in FIG. 3. The C—F distance population centered around 1.4 Å represents the C—F semi-ionic bonds. FIG. 4 shows several illustrations of the simulations of H₂ adsorption in the nitrogen-containing graphite fluoride. The H₂ molecules are highly mobile but display an attraction to the fluoride anions as shown in the RDF by the broad distribution of F—H distances with a mean at 2.5 Å. The calculated average total electronic energies of the nitrogen-containing graphite fluoride before and after H₂ adsorption are −343.90 eV and −505.41 eV, respectively. This corresponds to a heat of adsorption of −1.21 kcal/mole H₂ for the simulation of nitrogen containing graphite fluoride intercalation compound with 24 H₂/unit cell (8 wt. % H₂, C₂₄N₈F₈H₄₈).

Additional simulations of H₂ storage were performed with a lower 4 wt. % H₂ loading in the nitrogen containing graphite fluoride intercalation compound. The structure of the optimized unit cell is similar to FIG. 2 but with cell parameters as shown in Table 3. The calculated average total electronic energies of the F-intercalated graphite intercalation complex before and after H₂ adsorption are −343.90 eV and −425.65 eV, respectively. The total electronic energy of H₂ is −6.677 eV, leading to a calculated average H₂ adsorption energy in the system of −3.13 kcal/mol H₂.

### EXAMPLE 3

### Calculations of Hydrogen Interactions with a Graphite Fluoride Intercalation Compound

The unit cell selected to simulate H₂ adsorption in the graphite fluoride intercalation compound of formula C₃₂F₈, which does not contain nitrogen, was first fully equilibrated with and without adsorbed H₂ molecules. The formula and unit cell was chosen for providing a comparison with the nitrogen containing graphite fluoride intercalation compound C₂₄N₈F₈, with the N atoms being replaced with carbon atoms. The optimized unit cell parameters obtained from the simulations of H₂ adsorption in the graphite fluoride intercalation compound of formula C₃₂F₈ are shown in Table 4.

TABLE 4
Table 4. The calculated unit cell parameters for the graphite fluoride
intercalation compound with 12 H₂/unit cell (C₃₂F₈H₂₄),
and the graphite fluoride intercalation compound with
24 H₂/unit cell (C₃₂F₈H₄₈).

| | a | b | c | α | β | γ |
|---|---|---|---|---|---|---|
| C₃₂F₈H₂₄ | 9.734 | 9.734 | 6.556 | 89.7 | 89.9 | 120.2 |
| C₃₂F₈H₄₈ | 9.738 | 9.738 | 7.713 | 90.1 | 89.9 | 119.5 |

The calculated average total electronic energies of the graphite fluoride intercalation compound of formula C₃₂F₈H₄₈ before and after H₂ adsorption are −351.89 eV and −512.8 eV, respectively. Our calculation of the total electronic energy of H₂ in the simulation of formula C₃₂F₈H₄₈ is −6.677 eV, leading to a calculated average H₂ adsorption energy in the system of −0.64 kcal/mol H₂. This heat of adsorption is significantly lower than the −1.21 kcal/mole H₂ for the simulation of nitrogen containing graphite fluoride intercalation compound with 24 H₂/unit cell (C₂₄N₈F₈H₄₈).

Additional simulations of H₂ storage were performed with a lower, approximately 4 wt. % H₂ loading in the graphite fluoride intercalation compound. The optimized unit cell parameters are shown in Table 4. The calculated average total electronic energies of the graphite fluoride intercalation compound of formula C₃₂F₈H₂₄ before and after H₂ adsorption are −351.89 eV and −433.1 eV, respectively. The calculation of the total electronic energy of H₂ is −6.677 eV, leading to a calculated average H₂ adsorption energy in the system of −2.09 kcal/mol H₂. This heat of adsorption is lower than the −3.13 kcal/mole H₂ for the simulation of nitrogen containing graphite fluoride intercalation compound with 12 H₂/unit cell (C₂₄N₈F₈H₂₄).

### EXAMPLE 4

### Calculations of Hydrogen Interactions with a Nitrogen Containing Graphite Acetylide Dianion Intercalation Compound

Simulations of H₂ interactions within the nitrogen containing graphite acetylide dianion [(C≡C)²⁻] intercalation compound (represented by the molecular formulas C₁₁₂N₁₆(C₂)₈ or C₁₂₈N₁₆) were conducted and the fully equilibrated unit cell parameters with and without adsorbed H₂ are shown in Table 5. The molecular dynamics simulation showed that while some H₂ molecules remain intact during the duration of the simulation, there are hydrogen molecules that react with the anion to cleave the H—H bond, forming carbon-hydrogen bonds. The simulation yielded the average total energies for the material with and without H₂ uptake of −1600.17 eV and −1396.31 eV, respectively. This represents an average energy, representing all reactions of hydrogen within the lattice, of about −2.7 kcal/mol H₂ for a 3.5 wt. % hydrogen capacity.

TABLE 5
Table 5. The calculated unit cell parameters for the nitrogen containing
graphite acetylide dianion intercalation compound (C₁₂₈N₁₆) and
the nitrogen containing graphite acetylide dianion intercalation compound
with 32 H₂/unit cell (C₁₂₈N₁₆H₆₄).

| | a | b | c | α | β | γ |
|---|---|---|---|---|---|---|
| C₁₂₈N₁₆ | 19.347 | 19.368 | 4.797 | 90.0 | 90.0 | 119.9 |
| C₁₂₈N₁₆H₆₄ | 19.329 | 19.343 | 6.287 | 89.9 | 90.1 | 119.9 |

### EXAMPLE 5

### Calculations of Hydrogen Interactions with a Nitrogen Containing Graphite (N═C═N)²⁻ Intercalation Compound

Unit cell structures with and without H₂ adsorption in the nitrogen containing graphite (N═C═N)²⁻ intercalation compound (represented by the molecular formulas C₁₁₂N₁₆(CN₂)₈ or C₁₂₈N₃₂) were fully equilibrated prior to the MD simulations with the unit cell parameters shown in Table 6. Upon H₂ intercalation, an additional increase in interlayer distances between the graphitic sheets is observed due to H₂ interactions with the (N═C═N)²⁻ at both ends of the intercalated anions. The molecular dynamics simulation showed that while some H₂ molecules remain intact during the duration of the simulation, there are hydrogen molecules that react with the anion to cleave the H—H bond, forming carbon-hydrogen bonds. The total average energies of the nitrogen containing graphite (N═C═N)²⁻ intercalation compound with and without H₂ adsorption are −1691.50 eV and −1684.14 eV, respectively. This represents an average energy, representing all reactions of hydrogen within the lattice, of about −5.3 kcal/mol H₂.

TABLE 6
Table 6. The calculated unit cell parameters for the nitrogen containing
graphite (N═C═N)²⁻ intercalation compound (C₁₂₀N₃₂)
and the nitrogen containing graphite (N═C═N)²⁻ intercalation
compound with 32 H₂/unit cell (C₁₂₀N₃₂H₆₄).

| | a | b | c | α | β | γ |
|---|---|---|---|---|---|---|
| C₁₂₀N₃₂ | 19.274 | 19.279 | 5.627 | 89.9 | 90.2 | 120.8 |
| C₁₂₀N₃₂H₆₄ | 19.275 | 19.262 | 7.320 | 90.1 | 90.1 | 119.9 |

### EXAMPLE 6

### Hydrogen Adsorption Isotherms and Derived Isosteric Heats of Adsorption for a Graphite Fluoride Intercalation Compound

A mixture of F₂ and BF₃ gas containing a F₂:BF₃ molar ratio of 1:2 was prepared in a stainless steel ballast using 20% F₂ (in N₂) gas and BF₃ (purified by trap to trap distillation). The distillation was performed in doses to mix the appropriate ratio of BF₃ into the ballast already containing F₂. The ballast was allowed to equilibrate overnight to achieve thorough mixing of gases. Synthetic graphite powder (Alfa Aesar, 300 mesh or Timrex® SFG6) was dried at 900° C. for 3 hours under flowing argon. The graphite was then placed in a fluoropolymer tube reactor of approximately 1 inch diameter and 12 inch length dimensions in an argon-filled glove box. The fluoropolymer reactor was connected to a stainless steel manifold and evacuated to about 10⁻² torr pressure. The F₂/BF₃ mixture was slowly dosed into the fluoropolymer reactor at pressures up to 1000 torr. The reactor was mechanically agitated for 4 hours under the F₂/BF₃ mixture. The reactor was vented and evacuated until minimal outgassing from the sample was observed. This pressurization/agitation/evacuation procedure was repeated four times. The final evacuation of the graphite fluoroborate was performed for 4 hours to allow complete removal of BF₃ gas adsorbed to the graphite surface. Synthesis of a graphite fluoride from graphite fluoroborate was achieved by heating the graphite fluoroborate to 150-200° C. under a vacuum. More reproducible decomposition of graphite fluoroborate to graphite fluoride intercalation compound was achieved by using the 6 micron particle size SFG6 graphite. The graphite fluoroborate and graphite fluoride intercalation compound were characterized by X-ray powder diffraction and elemental analysis. The graphite fluoride was transferred under a purified argon atmosphere in a glovebox to a metal cell for adsorption analysis. Hydrogen isotherms were measured using differential pressure adsorption measurements. This technique quantifies the adsorption of gases by measuring the pressure difference between a cell containing an adsorbent and an identical reference cell (Zielinski, J. M.; Coe, C. G.; Nickel, R. J.; Romeo, A. M.; Cooper, A. C.; Pez, G. P. Accurate Hydrogen Sorption Measurements via Differential Pressure Analyses. Adsorption 2007, 13, 1). Hydrogen isotherms were measured at several temperatures (273, 298, and 313 K) at pressures up to 1500 psia (FIG. 5). The isosteric heat of adsorption (heat of adsorption at equal H₂ coverage) was subsequently determined from the isotherm data (FIG. 6).

### COMPARATIVE EXAMPLE 1

### Hydrogen Adsorption Isotherms on Activated Carbon

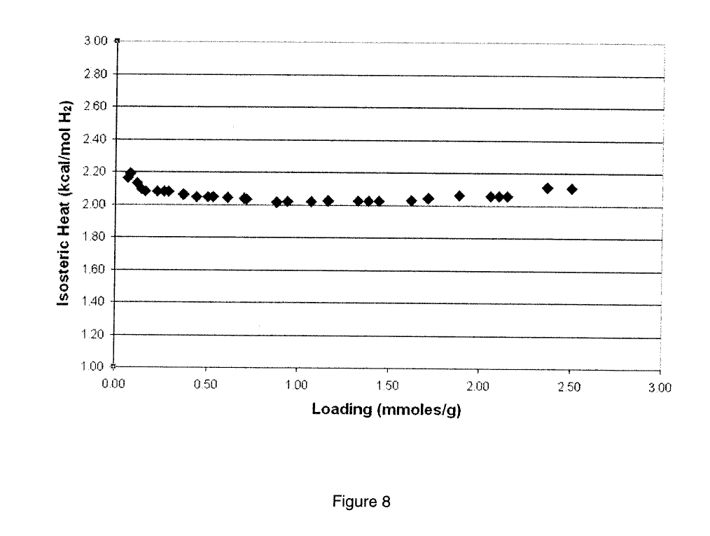

Activated carbon (Amoco® GX-31) was activated prior to adsorption measurements by heating at 300° C. under vacuum for 2 hours. The activated carbon was transferred under a purified argon atmosphere in a glovebox to a metal cell for adsorption analysis. Hydrogen isotherms were measured using differential pressure adsorption measurements as described in Example 7. Hydrogen isotherms were measured at several temperatures (273, 298, and 323 K) at pressures up to 1600 psia (FIG. 7). The isosteric heat of adsorption (heat of adsorption at equal H₂ coverage) was subsequently determined from the isotherm data (FIG. 8).

### Discussion of the Examples

Example 1 illustrates that hydrogen can interact with anions. The results shown in Table 1 below indicate that the anionic species in the gas phase are capable of adsorbing several H₂ molecules simultaneously with relatively high average energy. In addition, the results obtained with different levels of theory are generally consistent. For comparison purposes, we also calculated the H₂ interaction energy with a neutral gas-phase CsF molecule. The calculated average adsorption energy is −3.9 kcal/mol H₂, which is consistent with the cited qualitative experimental observations by Sweany et al. of a CsF and H₂ adduct in a solid argon matrix at 10 K.

Example 2 demonstrates that nitrogen containing graphite fluoride intercalation compounds are effective at storing hydrogen via interactions between hydrogen molecules and the intercalated fluoride ions. The equilibrated structures of the nitrogen containing graphite fluoride suggests that the F⁻ do not retain purely ionic character; instead, they interact with the graphene sheets by forming C—F semi-ionic bonds, which are nearly uniformly distributed between the graphitic sheets. The semi-ionic C—F bond formation leads to lattice expansion of this material, which then renders the interlayer spaces of the lattice accessible by H₂ molecules. There are indications of interactions between H₂ and F⁻ as the RDF of the F—H distance in the nitrogen containing graphite fluoride intercalation compound with 24 H₂/unit cell (C₂₄N₈F₈H₄₈) shows that H₂ molecules approach the fluorine atoms at distances as close as 1.5 Å, although the highest population of H—F distances is about 2.5 Å. Our calculation of the total electronic energy of H₂ is −6.677 eV, leading to a calculated average H₂ adsorption energy in the system of −1.21 kcal/mol H₂ for the simulation of the nitrogen containing graphite fluoride intercalation compound with 24 H₂/unit cell (C₂₄N₈F₈H₄₈). Note that this is the average adsorption energy of H₂ at near-ambient temperature for a relatively high H₂ loading of 8 wt. %. While this energy is considerably lower than the previously calculated gas-phase H₂—F⁻ adsorption energy, it is higher than what is found for H₂ in graphite at 77 K for a H₂ loading of approximately 1 wt. % (ca. −1 kcal/mol H₂; Pace, E. L.; Siebert, A. R. Heat of Adsorption of Parahydrogen and Orthodeuterium on Graphon, J. Phys. Chem. 1959, 63, 1398), thereby indicating that the F-intercalated graphite intercalation complex may be used as a H₂ storage material. Compared to 8 wt. % H₂ loading, the near-ambient temperature average H₂ adsorption energy at 4 wt. % loading is higher, −3.13 kcal/mol H₂.

Example 3 illustrates that graphite intercalation complexes with no nitrogen in the graphene sheets can also adsorb hydrogen, however, the enthalpy for hydrogen adsorption is smaller than hydrogen adsorption in comparable nitrogen containing graphite intercalation compounds. Similar to what is observed for the nitrogen containing graphite fluoride intercalation compound, the equilibrated structure indicates that the F do not retain purely ionic character; instead, they interact with the graphene sheets by forming C—F semi-ionic bonds, which are nearly uniformly distributed between the graphitic sheets. The F—H distance for the simulations of the graphite fluoride intercalation compound with 24 H₂/unit cell (C₃₂F₈H₄₈) indicates that H₂ molecules approach the fluorine atoms at distances as close as 1.8 Å, although the highest population of H—F distances is about 2.4 Å. Upon H₂ adsorption, the lattice was found to undergo expansion to accommodate the high density of H₂ molecules (about 8.2 wt. % of H₂). The relatively low energy of adsorption, −0.64 kcal/mol H₂, is reflective of the relatively hydrogen loading of the simulation and the absence of nitrogen in the graphene layers. A higher heat of adsorption, −2.09 kcal/mol H₂, is observed for the simulation of graphite fluoride intercalation compound with 12 H₂/unit cell (C₃₂F₈H₂₄).

Example 4 illustrates that graphite intercalation complexes of anions other than fluoride can be used for hydrogen storage. The interlayer distance between the graphene sheets is relatively large due to the orthogonal orientation of the acetylide dianions that form semi-ionic bonds with the graphitic sheets. The molecular dynamics simulation showed that while some H₂ molecules remain intact during the duration of the simulation, there are hydrogen molecules that react with the anion to cleave the H—H bond, forming carbon-hydrogen bonds. H₂ adsorption causes additional lattice expansion primarily due to H₂ molecules interacting at the termini of the acetylide dianions. The calculated average energy of −2.7 kcal/mol H₂ (for a 3.5 wt. % hydrogen capacity) is lower than that observed in the gas-phase calculations for the acetylide dianion but higher than the adsorption energy of hydrogen on non-intercalated graphite.

Example 5 demonstrates that graphite intercalation complexes in which the anions interact less strongly with the graphene layers have relatively high calculated hydrogen adsorption energies. Unlike what is observed for graphite intercalation complexes with the fluoride and (C≡C)²⁻ anions, the (N═C═N)²⁻ anions do not appear to form irreversible semi-ionic bonds with the graphitic sheets. Table 6 shows that the large size of the asymmetric (N═C═N)²⁻ anions forces the graphene sheets to separate to a greater interlayer distance than observed for the F⁻ and (C≡C)²⁻ intercalated graphitic compounds, which should allow facile access to the interlayer spaces for H₂ molecules. The molecular dynamics simulation showed that while some H₂ molecules remain intact during the duration of the simulation, there are hydrogen molecules that react with the anion to cleave the H—H bond, forming carbon-hydrogen bonds. The relatively high energy of −5.3 kcal/mol H₂ derived from the simulation is partially a result of the observed N—H and C—H bond formation.

Example 6 illustrates that graphite intercalation complexes adsorb hydrogen at near-ambient temperatures and display relatively high hydrogen adsorption enthalpies. The experimentally derived hydrogen isotherms of the graphite fluoride intercalation compound were used to calculate an isosteric heat of adsorption. The isosteric heat of adsorption is higher than the heat of adsorption on non-intercalated carbon materials such as graphite (ca. −1 kcal/mol H₂; Pace and Siebert) or activated carbon (ca. −2 kcal/mol H₂; Comparative Example 1).

Comparative Example 1 demonstrates that non-intercalated carbon materials can adsorb hydrogen but display relatively low heats of adsorption in comparison to the graphite fluoride intercalation compound. The isotherms at near ambient temperatures on an activated carbon show an isosteric heat of adsorption of only ca. −2 kcal/mol H₂. This low heat of adsorption limits the effective hydrogen capacity of activated carbon at useful, near-ambient temperatures.

This invention has been described with reference to particular aspects. It is understood that the description is not meant to be limiting, and modifications to this invention can be made that would fall within the scope of the claims which follow.

1) A compound comprising a graphitic structure and at least one anion having a volumetric charge density in the range of about 0.14 to about 0.02 electrons/Å³

2) The compound of claim 1 wherein said graphitic structure further comprises nitrogen.

3) The compound of claim 1 wherein the anion comprises at least one member selected from the group consisting of fluoride, diacetylide and cyanamide.

4) The compound of claim 1 wherein the graphitic structure has a lattice spacing that is greater than a graphitic structure without the anion.

5) The compound of claim 1 further comprising hydrogen.

6) The composition of claim 1 wherein the graphitic structure comprises at least one member selected from the group consisting of graphite, single walled carbon nanotubes, multiwalled carbon nanotubes, graphite nanofibers, carbon nanohorns, and boron nitride.

7). The composition of claim 1 wherein the anion comprises at least one member selected from the group consisting of O²⁻, NH²⁻, S²⁻, OH⁻, CN₂₋, NHLi⁻ and C₂²⁻.

8). The composition of claim 7 wherein the anion comprises CN₂²⁻

9). The composition of claim 7 wherein the anion comprises NH²⁻

10). The composition of claim 1 wherein the anion comprises at least one member selected from the group consisting of O²⁻, NH²⁻, S²⁻, OH⁻, CN₂⁻, NHLi⁻, C₂²⁻, CO₃²⁻, O₂⁻, and CN.

11) A method for storing hydrogen comprising contacting the composition of claim 1 with hydrogen at a pressure of about 1 to about 100 atm and at a temperature of about 0 degree C. to about 200 degree C.

12) A method for releasing hydrogen stored in the composition of claim 5 comprising lowering the pressure from about 100 to about 10 atm and at temperature of about 0 to about 200 degree C.

13). A method for reversibly storing hydrogen comprising:
contacting a solid-state material with hydrogen under conditions sufficient to retain at least a portion of hydrogen within the material; wherein the material comprises a graphitic structure comprising at least one intercalated anion.

14). The method of claim 13 wherein anion comprises at least one anion having a volumetric electron density from about 0.1 electron/Å 3 to about 0.018 electron/Å 3.

15). The method of claim 13 wherein the electron density is about from 0.1 electron/Å 3 to about 0.046 electron/Å 3.

16). The method of claim 13 wherein the anion comprises at least one member selected from the group consisting of O²⁻, NH₂⁻, S₂⁻, F⁻, OH⁻, CN₂²⁻, NHLi⁻, C₂²⁻, HF₂₋, CO₃²⁻, O₂⁻, CN⁻, BF₄⁻, SO₃F⁻, AsF₆⁻, and SbF₆⁻.

17). The method of claim 16 wherein the anion comprises F—.

18). The method of claim 13 wherein the graphitic host structure comprises an acceptor type graphite intercalation compound.

19). The method of claim 13 wherein method comprises at least one process selected from the group consisting of pressure-swing, temperature-swing, and combined temperature and pressure swing processes.

20). The method of claim 13 further comprising releasing at least a portion of the hydrogen retained in the material.

21). The method of claim 17 wherein the F— is ionic or semi-ionic.

22). A composition comprising: a material comprising at least one anion and said anion has a volumetric charge density in the range of about 0.14 to about 0.02 electrons/Å³ wherein molecular hydrogen is adsorbed within the material.
